SEQUENCE LISTING (4 sequences)

SEQ ID NO: 1 - DNA, 25 bp, Artificial Sequence
  Sequence is synthesized
cgtatcgcctccctcgcgccatcag

SEQ ID NO: 2 - DNA, 25 bp, Artificial Sequence
  Sequence is synthesized
ctatgcgccttgccagcccgctcag

SEQ ID NO: 3 - DNA, 16 bp, Artificial Sequence
  Sequence is synthesized
gtgccagcmgcngcgg

SEQ ID NO: 4 - DNA, 14 bp, Artificial Sequence
  Sequence is synthesized
gggttngntcgttg

(19) United States
(12) Patent Application Publication
(10) Pub. No.: US 2015/0209383 A1
(43) Pub. Date: Jul. 30, 2015

(54) METHODS AND COMPOSITIONS FOR MODULATING GASTROINTESTINAL BACTERIA TO PROMOTE HEALTH

(71) Applicant: GENERAL MILLS, INC., Minneapolis, MN (US)

(72) Inventors: Thomas W. Boileau, Plymouth, MN (US); Jennifer Brulc, Minneapolis, MN (US); Ravindranath Sreedhar Menon, Rogers, MN (US)

(21) Appl. No.: 14/355,963

(22) PCT Filed: Nov. 1, 2012
(86) PCT No.: PCT/US12/63008

§ 371 Date: May 2, 2014

Related U.S. Application Data
(60) Provisional application No. 61/555,800, filed on Nov. 4, 2011.

(51) Int. Cl. A61K 31/715

(52) U.S. Cl. CPC A61K 31/715

(57) ABSTRACT

Methods and compositions for treating medical conditions or improving health in which the composition is ingested in an amount sufficient to modify the proportion of bacteria in a gut microbiome of the individual ingesting the composition. The composition may comprise polydextrose or soluble com fiber, for example. The proportion of Coriobacteriaceae may be decreased to treat conditions such as elevated serum triglycerides, nonalcoholic fatty liver disease, metabolic syndrome, obesity, or type-2 diabetes. The proportion of V eillonellaceae may be increased to decrease serum cholesterol. The proportion of Faecalibacterium may be increased to treat inflammatory bowel disease or Chrohn's disease.

### BACKGROUND

Dietary fiber is believed to have numerous beneficial health effects. However, despite its health benefits, many people consume less than the recommended daily amount of fiber. In some cases, people may consume less than the recommended amount of fiber due to perceived or actual gastrointestinal intolerance. In other cases, the lack of adequate fiber intake may be due to taste preferences or lack of awareness. It would therefore be useful to include increased amounts of fiber in foods to assist people in increasing their daily fiber consumption, if the selected increased fiber can be tolerated and if the selected fiber does indeed produce health benefits.

One mechanism by which fiber can have beneficial health effects is by its effect upon the gastrointestinal microbiome, which is the microorganisms and the genetic elements of the microorganisms colonizing the gastrointestinal tract of an individual host. Some fibers are known to impact the gastrointestinal microbiome, namely bacteria, and to increase the levels of certain gastrointestinal bacteria and decrease the levels of others in ways which can be beneficial to the health of the host. Non-digestible fibers which support the increase in desirable gastrointestinal bacteria are referred to as prebiotics. In many cases, the actual effect of particular types of fiber upon the microbiome is unknown. In some cases, the effect of a fiber may be known only at a high taxonomic level, such as at the pylum or class level of bacteria. However, within the phylum or class may be bacteria which are beneficial as well as those which are detrimental to health. A more specific understanding of the effects of fibers upon the microbiome would therefore be useful, as well as a way to specifically effect the levels of particular bacterial families, genera or species.

Two fibers about which only limited information is known regarding their effect upon the gastrointestinal microflora are polydextrose and soluble corn fiber. Polydextrose, or PDX, is a synthetic soluble fiber which is a polymer of glucose. Soluble corn fiber, or SCF, is a commercially available product made from corn starch. Polydextrose and soluble corn fiber are food ingredients which are not well digested and are considered to be dietary fibers. However, their effect upon the microbiome is largely unknown.

### BRIEF DESCRIPTION OF THE FIGURES

The following figures are illustrative of particular embodiments of the present invention and therefore do not limit the scope of the invention. The figures are intended for use in conjunction with the explanations in the following detailed description. Embodiments of the present invention will hereinafter be described in conjunction with the appended photographs, wherein like numerals denote like elements.

FIG. 1 is a graph of bacterial families present in the stool of individuals in three experimental groups, in which stars indicate statically significant changes; and

FIG. 2a is a score plot and FIG. 2b is a loading plot of bacterial families and metabolites in the stool of the individuals in the three experimental groups.

### SUMMARY

Embodiments of the invention include compositions including polydextrose and/or soluble corn fiber for modulation of gastrointestinal bacteria and methods of using the compositions for health benefits or for the treatment of medical conditions. The modulation of the bacteria can provide health/medical benefits to the individual consuming the composition and can be used for the treatment of a medical condition and for maintaining a healthy state.

Embodiments of the invention include compositions for increasing or decreasing a proportion of a bacteria, the composition including polydextrose or soluble corn fiber. For example, in some embodiments, the composition is for decreasing a proportion of Coriobacteriaceae or increasing a proportion of Veillonellaceae or Faecalibacterium in a gut microbiome of an individual ingesting such composition, the composition including polydextrose or soluble corn fiber. The composition may decrease the proportion of Coriobacteriaceae by at least about 49 percent, such as between about 49 and 87 percent. The proportion of Veillonellaceae may be increased by at least about 50 percent, such as between about 50 percent and about 150 percent. The Faecalibacterium may include F. prausnitzii, for example. The proportion of Faecalibacterium may be increased by at least 2 percent, such as between about 2 percent and about 35 percent. In some embodiments, the composition decreases the proportion of Coriobacteriaceae and increases the proportion of Veillonellaceae and Faecalibacterium in the gut microbiome of the individual ingesting such composition.

In some embodiments the methods of treating a medical condition include ingesting a composition in an amount sufficient to decrease a proportion of Coriobacteriaceae in a gut microbiome of an individual ingesting the composition, wherein the composition comprises polydextrose or soluble corn fiber and wherein the medical condition comprises elevated serum triglycerides, nonalcoholic fatty liver disease, metabolic syndrome, obesity, or type-2 diabetes. The proportion of Coriobacteriaceae may be decreased by at least about 49 percent, such as between about 49 percent and about 87 percent.

Other embodiments include methods of decreasing serum cholesterol comprising ingesting a composition in an amount sufficient to increase a proportion of Veillonellaceae in a gut microbiome in an individual ingesting the composition, wherein the composition comprises polydextrose or soluble corn fiber. The proportion of Veillonellaceae may be increased by at least about 50 percent, such as between about 50 percent and about 150 percent.

Still other embodiments include methods of treating inflammatory bowel disease or Crohn's disease comprising ingesting a composition in an amount sufficient to increase a proportion of Faecalibacterium, wherein the composition comprises polydextrose or soluble corn fiber. The proportion of Faecalibacterium may be increased by at least 2 percent, such as between about 2 percent and about 35 percent. The Faecalibacterium may include F. prausnitzii, for example. The proportion of F. prausnitzii may be increased by at least about 15 percent, such as between about 15 percent and about 60 percent. Other bacteria may be increased or decreased according to embodiments of the invention for the treatment of these or other medical conditions.

In some embodiments, the composition may be a food, beverage, medication, or a dietary supplement. For example, the composition may be a cereal or a snack bar, and the cereal or snack bar may include soluble corn fiber. The composition may include between about 10 percent and about 40 percent polydextrose or soluble corn fiber, for example. The amount of the composition may be consumed at least one time per day, such as three times per day. In some embodiments, the amount of the composition includes between about 7 grams and 21 grams of soluble corn fiber per day. The amount of the composition may be consumed daily for at least about 1 day, such as between about 1 and about 21 days.

### DETAILED DESCRIPTION OF ILLUSTRATIVE EMBODIMENTS

The following detailed description is exemplary in nature and is not intended to limit the scope, applicability, or configuration of the invention in any way. Rather, the following description provides practical illustrations for implementing exemplary embodiments of the present invention. Examples of materials and processes are provided for selected elements, and all other elements employ that which is known to those of skill in the field of the invention. Those skilled in the art will recognize that many of the examples provided have suitable alternatives that can be utilized.

The applicants have discovered that polydextrose and soluble corn fiber can both be used to produce significant changes in the microbial population of the gut. These fibers can be consumed to obtain these microbial shifts and the associated health benefits. Embodiments of the invention therefore include methods and compositions for modulating levels of gastrointestinal bacteria. The composition may be a food or beverage product, a medication, or any other consumable, and may include polydextrose, soluble corn fiber, or a combination of both polydextrose and soluble corn fiber.

The polydextrose which may be used in various embodiments is a synthetic polymer of glucose. It is a highly branched, randomly linked polysaccharide of glucose with a degree of polymerization between 3-10 and different combinations of α and β glycosidic linkages. Polydextrose is commercially available from various sources, including from Tate & Lyle of as PROMOTOR™ and may be used in a powdered form, as an aqueous solution, or any other form. One method which can be used for making polydextrose is described in U.S. Pat. No. 3,766,165, the disclosure of which is hereby incorporated by reference.

Soluble corn fiber, also referred to as soluble maize fiber, is made from corn starch and contains oligosaccharides with random glycosyl bonds and may contain minor amounts of monosaccharides. Soluble corn fiber is commercially available from various sources including from Matsutani America, Inc of Itasca Ill. as Fibersol®-LQ.

Embodiments of the invention include foods products (including beverages), medications, and dietary supplements, including polydextrose and/or soluble corn fiber, which can be consumed in amounts sufficient to modulate bacteria levels. Foods which may be used in embodiments of the invention may include foods which typically contain significant sources of fiber such as grain based foods including bread and bread products (such as rolls, bagels, buns, pitas), baked goods (such as cookies, cakes, bars, muffins, brownies, biscuits), ready to eat cereals, cooked cereals such as oatmeal, cereal bars, meal replacement bars, snack bars, waffles, pancakes, pancake mixes, pizza crust and pizza rolls, pie crust, pasta, crackers, tortillas, chips (such as potato, corn, pita), pretzels, dough (such as for bread products, pizza, baked goods), and flour, as well as non-grain based foods such as soups, salsas, sauces such as pizza or spaghetti sauce, jams and jellies, frozen fruit bars, apple sauce or other fruit sauces, and relish, for example. Other foods include foods which do not typically include significant levels of fiber, or typically include only low amounts of fiber, such as dairy products including cheese (including natural, processed, or artificial), yogurt, ice cream, frozen yogurt, candy, and chocolate, for example. Beverages which may be used in embodiments of the invention include fruit and vegetable juices, dairy drinks such as milk, yogurt drinks, smoothies, malts and shakes, hot chocolate, and sodas, sports drinks, vitamin and drinks, for example.

Polydextrose and/or soluble corn fiber may be included in the food product as the only source of fiber or as an additional source of fiber. In some embodiments, the polydextrose and/or soluble corn fiber may be used in addition to the fiber normally present in the food product. Alternatively, the amount of fiber from other sources may be reduced in whole or in part and the polydextrose and/or soluble corn fiber may be included in an amount that partially or completely replaces the other fiber source, and may or may not also provide additional fiber beyond the replacement amount.

The amount of soluble corn fiber and/or polydextrose included in the food product may depend on the type of food product in which it is used. For example, the amount of soluble corn fiber and/or polydextrose included in a product which is typically high in fiber such as cereal or snack bars may be between about 5 percent and about 40 percent, such as about 10 percent to about 40 percent or about 10 percent to about 30 percent. In some embodiments, the food product is a snack bar and includes about 15 percent to about 25 percent soluble corn fiber and/or polydextrose. In other embodiments, the food product is a ready-to-eat cereal and includes between about 3 percent to about 15 percent soluble corn fiber and/or polydextrose. Other food products, such as dairy products like yogurts, may include lower amounts of soluble corn fiber and/or polydextrose, such as about 0.5 percent to about 10 percent or about 1 percent to about 8 percent. In some embodiments, such products may include about 3-6 percent soluble corn fiber and/or polydextrose.

In some embodiments, the food product, medication, or dietary supplement may also include a probiotic agent, in addition to the polydextrose and/or soluble corn fiber. Probiotic agents include live bacteria which may have a beneficial impact on health. Examples of probiotic agents which may be used in various embodiments include lactic acid bacteria such as Lactobacillus bulgaricus, Lactobacillus acidophilus, Lactobacillus rhamnosus, Lactobacillus casei, Lactobacillus johnsonii, Lactobacillus plantarum, Lactobacillus thermophilus, Lactobacillus reuteri, and bifidobacteria such as bifidobacterium infantis and bifidobacterium animalis. Other probiotics which may be used include Bacillus coagulans, Saccharomyces boulardii, Saccharomyces, Streptococus, Enterococcus and Bacillus strains.

According to some embodiments, the polydextrose and/or soluble corn fiber may be consumed in an amount effective to achieve the desired result, such as a change in gastrointestinal bacteria levels and/or a health benefit. In some embodiments, that amount is between about 1 g/day and about 50 g/day, or between about or between about 7 g/day and about 21 g/day. In some embodiments, this daily amount may be consumed every day for at least about one day, two days, three days, four days, one week, two weeks, three weeks, or one month. The daily amount may be consumed once per day or may be divided into two or three portions that may or may not be equal. In some embodiments, the daily amount may be consumed with or as a component of a meal, such as with one, two or three meals, including breakfast, lunch and/or dinner, for example. For example, the daily amount may be consumed in full each day with breakfast and may be consumed as a breakfast food such as a ready to eat cereal. In other embodiments, all or a portion of the daily amount may be consumed between meals, such as in the form of a snack.

By consuming an adequate amount of polydextrose and/or soluble corn fiber, such as in a food, medication or dietary supplement, beneficial changes in the gut microbial population can be achieved. Embodiments of the invention may be effective at increasing the proportion of beneficial bacteria, decreasing the proportion of harmful bacteria, and/or beneficially improving the ratio of bacteria in the gastrointestinal system of an individual consuming the polydextrose and/or soluble corn fiber. As used herein, the proportion of a bacteria represents the percent of the bacteria compared to all measured bacteria, unless otherwise indicated. The proportion of a bacteria may be determined most easily by quantification of bacteria in the stool (such as by PCR and sequencing, including but not limited to pyrosequencing, 16S analysis, illumina sequencing, and sanger sequencing, for example), and as such represents the proportion in the gut overall. Alternatively, the proportion of a bacteria may be determined by sampling in the gut, but results may vary depending upon the location of the sample.

In some embodiments, the proportion of certain bacteria is decreased, such as Actinobacteria. In particular, the proportion of Bifidobacteriaceae and Coriobacteriaceae may be decreased.

In some embodiments, the proportion of certain bacteria is increased, such as Lactobacillaceae. Other bacteria which may be increased include Clostridiaceae, Beillonellaceae, and Verrucomicrobiaceae.

In some embodiments, the proportion of some families of bacterial in a phylum may be increased (relative to the phylum itself), while others may be decreased, such as bacteria within the phylum of Proteobacteria. For example, Hyphomicrobiaceae may be decreased, while Alcaligenaceae may be increased.

Certain genera of bacteria in the phylum Firmicutes may be increased, including Faecalibacterium, Phascolarctobacterium, Dialister, Clostridium, Akkermansia, and Lactobacillus. Other genera of bacteria in the phylum Firmicutes may be decreased, including Ruminococcus, Eubacterium, Dorea, Coprococcus, and Oscillospira.

Embodiments of the invention may be used to increase the proportion of Bifidobacterium spp. In some embodiments, the proportion of Bifidobacterium spp. may be raised by approximately 1 log, and this increase may be used to provide improved colonic health.

The modulation of bacteria levels resulting from embodiments of the invention may be used for health maintenance, for certain health/medical benefits as well as for maintenance of a health/medical benefit. For example, in some embodiments, the gastrointestinal proportion of Veillonellaceae is increased. Veillonellaceae is associated with the deconjugation of bile salts and acids in the gut, which makes the salts less soluble and results in lower absorption of the salts. By increasing the proportion of Veillonellaceae, the absorption of bile salts can therefore be decreased, which may result in lowering of serum cholesterol levels and may be used to treat or prevent elevated serum cholesterol. Veillonellaceae may be increased about 1.6-2.6 fold, or about 1.9-2.3 fold, for example. In some embodiments, it is increased by at least about 50 percent, such as about 50 percent to about 150 percent.

In some embodiments, the gastrointestinal proportion of bacteria of the family Coriobacteriaceae is decreased. Coriobacteriaceae are strongly linked with increased hepatic triglycerides, glycogen and glucose, and there is a correlation between certain bacteria of the Coriobacteriaceae family and non-HDL plasma cholesterol and cholesterol absorption. While not intending to be bound by theory, this correlation may relate to the ability of Coriobacteriaceae to transform bile acids and affect cholesterol metabolism through increasing enterohepatic circulation. Therefore, embodiments of the invention which decrease the proportion of Coriobacteriaceae may be used to lower serum cholesterol levels or to treat elevated serum cholesterol. In some embodiments, the proportion of Coriobacteriaceae may be decreased by about 2.9 to about 3.5 fold. In some embodiments, the proportion of Coriobacteriaceae may be decreased by at least about 49 percent, such as by about 49 percent to about 87 percent.

In some embodiments, the gastrointestinal proportion of Faecalibacterium is increased, in particular Faecalibacterium prausnitzii, which is known to have anti-inflammatory properties. The proportion of Faecalibacterium may be increased by about 1.02 fold to about 1.34 fold, or about 1.16 fold to about 1.23 fold. In some embodiments, it may be increased by at least about 2 percent, such as between about 2 percent to about 35 percent. The proportion of Faecalibacterium prausnitzii may therefore be increased for the prevention or treatment of gastrointestinal disorders involving inflammation, such as inflammatory bowel disease (IBD) and Chrohn's disease. The anti-inflammatory effect of Faecalibacterium prausnitzii may be mediated by decreased IFNγ production and upregulation of colonic PPARγ expression. Embodiments of the invention may therefore be used to prevent or treat gut inflammation by decreasing IFNγ production and upregulation of colonic PPARγ expression. In some embodiments, the proportion of Faecalibacterium prausnitzii may be increased by about 1.15 fold to about 1.58 fold, or about 1.3 fold to about 1.4 fold. In some embodiments, the proportion of Faecalibacterium prausnitzii may be increased by about 15 percent, such as between about 15 percent and about 60 percent.

Roseburia species, the proportion of which may be increased in embodiments of the invention, are associated with beneficial effects on the gut in healthy and diseased individuals. For example, Roseburia spp. have a high capacity to form conjugated linoleic acid from linoleic acid, which has health benefits. In addition, both Roseburia spp. and F. prausnitzii produce butyrate using acetate as a substrate, which also have effects on gut health. Embodiments of the invention may therefore be used to increase the proportion of Roseburia spp. and F. prausnitzii to improve gut health.

In some embodiments, the proportion of Veillonellaceae, Coriobacteriaceae and/or F. prausnitzii may be modulated to prevent or correct dysbiosis in certain diseases.

By modulating the proportion of certain gastrointestinal bacteria, embodiments of the invention can decrease the levels of putrefactive compounds in the stool. Such putrefactive compounds are linked to bowel cancer and can cause damage to the colonic epithelium and become tumor growth promotors. Therefore, by decreasing the levels of putrefactive compounds, embodiments of the invention can be used to decrease the risk of bowel cancer such as colon or rectal cancer. Putrefactive compounds which may be decreased by embodiments of the invention include ammonia, phenol, indoles, and branched chain fatty acids (isobytyrate, isovalerate, and valerate). Embodiments of the invention can also cause increased stool production, creating a dilution effect and thereby decreasing the contact between putrefactive compounds and the intestinal epithelium. In some embodiments, the fermentation metabolites may be adjusted by the consumption of polydextrose and/or soluble corn fiber. Various embodiments may therefore be used to counteract the adverse effects of protein fermentation.

### Experimental

A group of twenty-five healthy men (of whom twenty-one completed the study) were randomly divided into three groups: a control group; a supplemental polydextrose consuming group (the “PDX group”); and a supplemental soluble corn fiber consuming group (the “SCF group”). All participants consumed three snack bars per day, one with each meal, for 21 days. The control group's snack bars included no additional fiber. The PDX group's snack bars included 7 grams of polydextrose for a total of 21 grams of polydextrose per day. The SCF group's snack bars included 7 grams of soluble corn fiber for a total of 21 grams of soluble corn fiber per day.

The composition of the snack bars consumed by the participants is shown in Table 1 below. The polydextrose used in the bars was Litesse II®, obtained from Tate & Lyle Ingredients, Decatur, Ill., which had 1 kcal/gram. The soluble corn fiber used in the bars was Promitor® Soluble Corn Fiber 70, obtained from Tate & Lyle Ingredients, Decatur, Ill., which contained at least 70 percent soluble fiber (dsb, dry solid basis), a maximum of 20 percent sugar, and 2 kcal/gram.

TABLE 1

| | Control | PDX group | SCF group |
|---|---|---|---|
| | bar (%) | bar (%) | bar (%) |
| Corn Syrups | 70 | 22 | 0 |
| Polydextrose | 0 | 53 | 0 |
| Soluble Corn Fiber | 0 | 0 | 54 |
| Liquid Sugar/ | 17 | 8 | 36 |
| Maltodextrin | | | |
| Oils/Lecithin | 5 | 5 | 5 |
| Sugars | 2 | 7 | 0 |
| Glycerine | 5 | 4 | 4 |
| Flavors | 1 | 1 | 1 |

Other than the difference in fiber content, the three bars were chemically very similar. A comparison of the nutritional content of the snack bars consumed by the participants is shown in Table 2, below.

TABLE 2

| | Control group bar | PDX group bar | SCF group bar |
|---|---|---|---|
| Calories | 398/100 g | 381/100 g | 380/100 g |
| Carbohydrates | 78.6% | 81.7% | 80.0% |
| Moisture | 9.03% | 9.23% | 10.33% |
| Protein | 3.65% | 3.77% | 3.52% |
| Total Dietary Fiber | 0.90% | 0.60% | 1.40% |
| Insoluble Fiber | 0.70% | 0.20% | 0.30% |
| Soluble Fiber | 0.20% | 0.40% | 1.10% |
| Resistant | 0.10% | 18.60% | 16.70% |
| Oligosaccharides | | | |
| Water Activity | 0.489 | 0.475 | 0.531 |
| Fiber per bar (g) | 0.42 | 8.064 | 7.602 |

After a 16 day adaptation period, stool was collected during the final 5 days of the experiment for each group. Fresh samples were weighed, pH measurements were made, and a microbiota subsample was removed for bacterial DNA extraction. The fresh samples were then frozen at −20° C. and the microbiota subsamples were stored at −80° C.

### Example 1

Fecal principal component analysis was performed as described in B. M. Vester Boler et al., Digestive physiological outcomes related to polydextrose and soluble maize fibre consumption by healthy adult men, British Journal of Nutrition (2011) p. 1-8, the disclosure of which is hereby incorporated by reference in its entirety. The mean values of the results of these analyses are shown Table 3, below.

TABLE 3

| | Control | PDX | SCF | | |
|---|---|---|---|---|---|
| | Group | Group | Group | SEM | P |
| Endproduct (μmol/g | | | | | |
| DM faeces) | | | | | |
| Ammonia | 137.5 | 97.5 | 117.0 | 7.82 | <0.0001 |
| 4-Methyl phenol | 1.5 | 0.6 | 0.9 | 0.20 | 0.003 |
| 2,3-Methyl indole | 0.3 | 0.3 | 0.3 | 0.11 | 0.72 |
| Indole | 1.0 | 0.3 | 0.5 | 0.17 | 0.003 |
| Isobutyrate | 9.1 | 5.3 | 6.5 | 0.88 | <0.0001 |
| Isovalerate | 10.4 | 5.7 | 6.9 | 0.99 | <0.0001 |
| Valerate | 10.7 | 7.6 | 9.0 | 1.17 | 0.005 |
| Total BCFA | 30.3 | 18.6 | 22.4 | 2.76 | <0.0001 |
| Acetate | 320.3 | 272.3 | 351.6 | 24.95 | 0.02 |
| Propionate | 97.3 | 74.0 | 101.6 | 10.48 | 0.008 |
| Butyrate | 93.8 | 60.5 | 81.0 | 9.13 | 0.005 |
| Total SCFA | 511.4 | 407.0 | 534.0 | 41.96 | 0.018 |
| Ratio | | | | | |
| Acetate | 0.63 | 0.68 | 0.67 | 0.01 | <0.0001 |
| Propionate | 0.19 | 0.18 | 0.19 | 0.01 | 0.097 |
| Butyrate | 0.18 | 0.15 | 0.15 | 0.006 | 0.005 |

From the Table 3, above, it can be seen that fecal ammonia concentration was decreased in both the PDX and SCF groups, with the largest decrease in the PDX group. Similarly fecal valerate contractions were lower in both the PDX and SCF groups, with the largest decrease in the PDX group. Fecal 4-methylphenol, indole, isobutyralte, isovalerate and total branched chain fatty acid (BCFA) concentrations were decreased in both the PDX and SCF groups. Fecal acetate, propionate, and butyrate were decreased in the PDX group.

### Example 2

E. coli, Bifidobacterium genus and Lactobacillus genus in each of the feces samples were quantified by quantitative PCR analysis using specific primers. Amplification was performed in triplicate for each bacterial group within each sample. For amplification, 10 μl final volume containing 2×SYBR Green PCR Master Mix, 15 pmol of each primer and 5 ng of template DNA was used. Pure cultures of each bacterium were utilized to create a five-fold dilution series in triplicate from the target species. DNA from each serial dilution was amplified along with fecal DNA samples using a Taqman ABI PRISM 7900HT Sequence Detection System. The colony-forming units of each standard curve serial dilution was determined by plating each of the target bacteria. Cycle threshold values were plotted against the standard curves for quantification (in colony forming units per gram feces) of the target bacterial DNA from fecal samples. The results are shown in Table 4, below, in which the values represent the mean values of the log colony forming units per gram DM (dry matter) feces.

TABLE 4

| | Control | PDX | SCF | | |
|---|---|---|---|---|---|
| | Group | Group | Group | SEM | P |
| Bifidobacterium spp. | 6.9 | 7.7 | 8.2 | 0.61 | 0.03 |
| Lactobacillus spp. | 10.1 | 10.1 | 10.5 | 0.69 | 0.64 |
| Escherichia coli | 8.9 | 8.6 | 9.5 | 0.47 | 0.22 |

It can be seen that the concentrations of Bifidobacterium spp. were higher in both the PDX and SCF groups, with the greatest increase in the SCF group. Both the PDX group and the SCF group have an approximate 1 log increase in Bifidobacterium spp. There was no significant difference in Fecal Lactobacillus spp. or E. Coli concentrations between the groups.

### Example 3

Bacterial DNA extraction was done using a QIAamp DNA stool mini kit (Qiagen, Valencia, Calif.) using the repeated bead beating plus column method. Fecal DNA was quantified using a NanoDrop ND-1000 spectrophotometer (NanoDrop Technologies, Wilmington, Del.). Extracted DNA from the three fresh samples of each subject per collection period were pooled, diluted to 20 ng/μl and genomic DNA quality was assessed using electrophoresis using precast E-Gel® EX Gel 1% (Invitrogen, Carlsbad, Calif.). Amplification of the variable region V4-V6 of the 16S rRNA gene was done using barcoded primers. The barcoded primers contained forward ‘LinkerA’ sequence (5′-CGTATCGCCTCCCTCGCGCCATCAG) and reverse ‘LinkerB’ sequence (5′-CTATGCGCCTTGCCAGCCCGCTCAG), a multiplex identifier (MID) unique to each sample, and a Eubacterial-specific sequence for the V4-V6 region of the 16S rRNA gene. The 16S universal Eubacterial primers 530F (5′-GTGCCAGCMGCNGCGG) and 1100R (5′-GGGTTNGNTCGTTG) were used to amplify a 600-bp region of the 16S rRNA (24). PCR reactions were performed for each sample using a barcoded forward primer, barcoded reverse primer, dNTP mix, FastStart 10× buffer with MgCl₂, FastStart HiFi Polymerase and genomic DNA. PCR amplicons were further purified using AMPure XP beads (Beckman-Coulter, Inc., Brea, Calif.) and DNA concentration and quality were measured again. The amplicons were combined in equimolar ratios to create a DNA pool for pyrosequencing using a 454 genome sequencer and FLX titanium reagents (Roche Applied Science, Indianapolis, Ind.). Poor quality reads and primer dimers were removed.

The sequences were selected to estimate total bacterial diversity. A total of 4500+/−100 rarified sequences were selected from each sample based on highest average quality score and trimmed to 250 bp. Bacterial ID community structure was evaluated using Phred25 quality reads, including both 530F and 1100R oriented, trimmed then depleted of unwanted sequences. The final sequence data (500,588 total sequences, 8,600 for each subject) were evaluated using Kraken against a 01-11-11 version database curated from NCBI to include >350,000 high quality 16S bacterial and archaeal sequences as well as quality control sequences. Blast output based upon top hit designations were compiled to generate percentage files at each taxonomic level. Operational taxonomic unit (OTU) at 95 percent similarity, Abundance-based coverage estimation (ACE), and bias-corrected Chao 1 richness estimates of diversity and the Shannon Index were performed. No significant difference was found between the control group, the PDX group and the SCF group. As such, there was no difference in overall fecal bacterial diversity between the groups.

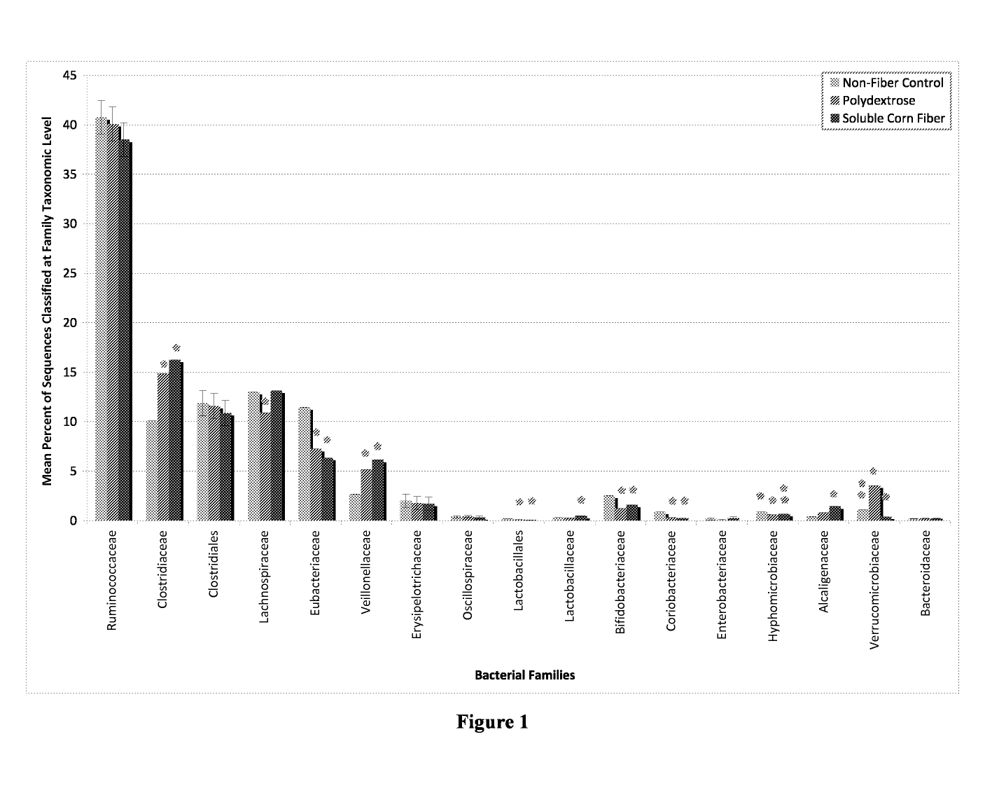

Table 5 below shows the bacterial phyla and families within the phyla (expressed as percentage of sequences) in the feces of each test group. The numbers shown represent the mean plus or minus standard error. This data is also shown graphically in FIG. 1 where the presence of a significant difference is indicated by an asterisk. It can be seen that the phylum Firmicutes was the most common in all groups, with no significant difference between the test groups. However, among the Firmicutes, Clostridiaceae, Veillonellaceae, and Lactobacillaceae increased in both the PDX and SCF groups. Lachnospiraceae decreased in only the PDX group, while Eubacteriaceae decreased in both the PDX and SCF groups.

The phyla Actinobacteria decreased in the PDX and SCF groups, with both Bifidobacteriaceae and Coriobacteriaceae families decreasing in both groups. The phyla of Proteobacteria increased only in the SCF group. However, among the Proteobacteria, Hyphomicrobiaceae decreased in the PDX group only, and Alcaligenaceae increased in the SCF group only. The phylum of Verrucomicrobia increased in both the PDX and SCF groups, with a larger increase in the PDX group.

TABLE 5

| | NFC | PDX | SCF | P¹ |
|---|---|---|---|---|
| Firmicutes | 93.23 ± 1.33 | 92.74 ± 1.36 | 94.47 ± 1.36 | 0.3966 |
| Ruminococcaceae | 40.74 ± 1.69 | 40.07 ± 1.71 | 38.49 ± 1.71 | 0.2157 |
| Clostridiaceae | 10.09 ± 1.18^{a} | 14.88 ± 1.19^{b} | 16.25 ± 1.19^{b} | <0.0001 |
| Clostridiales² | 11.87 ± 1.25 | 11.58 ± 1.26 | 10.87 ± 1.26 | 0.4419 |
| Lachnospiraceae | 12.98 ± 1.01^{b} | 10.89 ± 1.03^{a} | 13.13 ± 1.03^{b} | 0.0056 |
| Eubacteriaceae | 11.46 ± 1.19^{b} | 7.24 ± 1.20^{a} | 6.34 ± 1.20^{a} | <0.0001 |
| Veillonellaceae | 2.68 ± 0.68^{a} | 5.17 ± 0.69^{b} | 6.15 ± 0.69^{b} | <0.0001 |
| Erysipelotrichaceae | 2.01 ± 0.68 | 1.78 ± 0.68 | 1.71 ± 0.68 | 0.2153 |
| Oscillospiraceae | 0.39 ± 0.12 | 0.44 ± 0.12 | 0.34 ± 0.12 | 0.3989 |
| Lactobacillales³ | 0.22 ± 0.04^{b} | 0.10 ± 0.04^{a} | 0.08 ± 0.04^{a} | 0.0223 |
| Lactobacillaceae | 0.31 ± 0.11^{a} | 0.28 ± 0.11^{a} | 0.48 ± 0.11^{b} | 0.0230 |
| Actinobacteria | 3.45 ± 0.61^{b} | 1.55 ± 0.62^{a} | 1.86 ± 0.62^{a} | 0.0001 |
| Bifidobacteriaceae | 2.55 ± 0.57^{b} | 1.25 ± 0.58^{a} | 1.61 ± 0.58^{a} | 0.0019 |
| Coriobacteriaceae | 0.91 ± 0.14^{b} | 0.31 ± 0.14^{a} | 0.26 ± 0.14^{a} | <0.0001 |
| Proteobacteria | 1.74 ± 0.30^{a} | 1.75 ± 0.30^{a} | 2.82 ± 0.30^{b} | 0.0045 |
| Enterobacteriaceae | 0.19 ± 0.11 | 0.04 ± 0.11 | 0.27 ± 0.11 | 0.1106 |
| Hyphomicrobiaceae | 0.89 ± 0.17^{b} | 0.61 ± 0.17^{a} | 0.68 ± 0.17^{ab} | 0.0314 |
| Alcaligenaceae | 0.42 ± 0.29^{a} | 0.81 ± 0.29^{a} | 1.44 ± 0.30^{b} | 0.0004 |
| Verrucomicrobia | 1.08 ± 1.20^{ab} | 3.54 ± 1.22^{b} | 0.41 ± 1.22^{a} | 0.02 |
| Verrucomicrobiaceae | 1.08 ± 1.20^{ab} | 3.54 ± 1.22^{b} | 0.41 ± 1.22^{a} | 0.02 |
| Bacteroidetes | 0.45 ± 0.10 | 0.34 ± 0.10 | 0.43 ± 0.10 | 0.4196 |
| Bacteroidaceae | 0.19 ± 0.03 | 0.23 ± 0.03 | 0.23 ± 0.03 | 0.4932 |

¹ANOVA with Tukey post hoc test.
²Unknown family within order Clostridales.
³Unknown family within order Lactobacillales.
^{a,b}Means in the same row not sharing a common superscript letter are different (P < 0.05).

Table 6 below characterizes the bacteria by bacterial genera (expressed as percentage of sequences) for each group. The data shown represents the mean, plus or minus standard error. It can be seen that the proportion of Faecalibacterium, unknown genera within Clostridiaceae, Phascolarctobacterium and Dialister was all increased in both the PDX and the SCF groups. In contrast, Clostridium and Akkermansia increased only in the PDX group and Lactobacillus increased only in the SCF group. Ruminococcus, Eubacterium, Dorea, Coprococcus, Bifidobacterium, and Coriobacterium decreased in both the PDX and SCF groups. Oscillospira decreased in the SCF group only.

TABLE 6

| | Control Group | PDX Group | SCF Group | P¹ |
|---|---|---|---|---|
| Firmicutes | | | | |
| Faecalibacterium | 20.72 ± 2.25^{a} | 24.06 ± 2.78^{b} | 25.49 ± 2.28^{b} | 0.0022 |
| Ruminococcus | 13.08 ± 0.91^{b} | 9.22 ± 0.92^{a} | 7.68 ± 0.92^{a} | <0.0001 |
| Eubacterium | 11.83 ± 1.26^{b} | 7.55 ± 1.27^{a} | 6.87 ± 1.27^{a} | <0.0001 |
| Clostridiaceae² | 1.96 ± 0.98^{a} | 5.32 ± 0.99^{b} | 8.02 ± 0.99^{b} | <0.0001 |
| Clostridium | 8.11 ± 0.72^{a} | 9.50 ± 0.73^{b} | 8.26 ± 0.73^{a} | 0.0182 |
| Roseburia | 8.77 ± 0.97^{ab} | 7.42 ± 0.99^{a} | 9.78 ± 0.99^{b} | 0.0058 |
| Subdoligranulum | 4.42 ± 0.66 | 4.18 ± 0.66 | 3.84 ± 0.66 | 0.3379 |
| Oscillospira | 2.19 ± 0.56^{b} | 2.28 ± 0.57^{b} | 1.44 ± 0.57^{a} | 0.0113 |
| Phascolarctobacterium | 1.51 ± 0.50^{a} | 2.30 ± 0.50^{b} | 2.80 ± 0.50^{b} | 0.0002 |
| Akkermansia | 1.08 ± 1.19^{a} | 3.54 ± 1.22^{b} | 0.41 ± 1.22^{a} | 0.02 |
| Dorea | 1.40 ± 0.21^{b} | 0.69 ± 0.21^{a} | 0.72 ± 0.21^{a} | <0.0001 |
| Dialister | 0.97 ± 0.78^{a} | 2.35 ± 0.79^{b} | 2.87 ± 0.79^{b} | 0.0007 |
| Coprococcus | 0.58 ± 0.09^{b} | 0.42 ± 0.09^{a} | 0.36 ± 0.09^{a} | 0.0035 |
| Lactobacillus | 0.30 ± 0.11^{a} | 0.27 ± 0.11^{a} | 0.47 ± 0.11^{b} | 0.0241 |
| Actinobacteria | | | | |
| Bifidobacterium | 2.54 ± 0.57^{b} | 1.25 ± 0.57^{a} | 1.60 ± 0.57^{a} | 0.0019 |
| Coriobacterium | 0.76 ± 0.14^{b} | 0.23 ± 0.14^{a} | 0.22 ± 0.14^{a} | 0.0003 |
| Bacteroidetes | | | | |
| Bacteroides | 0.19 ± 0.03 | 0.23 ± 0.03 | 0.23 ± 0.03 | 0.4932 |

¹ANOVA with Tukey post hoc test.
²Unknown genera within family Clostridiaceae.
^{a,b}Means in the same row not sharing a common superscript letter are different (P < 0.05).

Table 7 below shows the data for the bacterial species (expressed as percentage of sequences). It can be seen that Faecalibacterium prausnitzii was increased in both the PDX and SCF groups. Eubacterium rectale, Eubacterium halii, Ruminococcus spp. and Bifidobacterium spp. were decreased in both the PDX and SCF groups. Roseburia spp. was increased only in the SCF group. Clostridium leptum was increased only in the PDX group.

TABLE 7

| | Treatment² | | | |
|---|---|---|---|---|
| | NFC | PDX | SCF | P¹ |
| Faecalibacterium spp.² | 12.15 ± 1.55 | 12.74 ± 1.56 | 13.48 ± 1.56 | 0.3789 |
| Faecalibacterium prausnitzii | 8.57 ± 1.51^{a} | 11.38 ± 1.52^{b} | 12.05 ± 1.52^{b} | <0.0001 |
| Clostridiales spp. | 10.98 ± 1.26 | 10.84 ± 1.27 | 10.04 ± 1.27 | 0.4406 |
| Clostridiaceae spp. | 1.96 ± 0.98^{a} | 5.32 ± 0.99^{b} | 8.02 ± 0.99^{b} | <0.0001 |
| Clostridium spp. | 5.57 ± 0.71 | 6.87 ± 0.72 | 5.57 ± 0.72 | 0.0595 |
| Clostridium leptum | 0.32 ± 0.12^{a} | 0.82 ± 0.12^{b} | 0.66 ± 0.12^{a} | <0.0001 |
| Ruminococcus spp. | 9.83 ± 0.74^{b} | 6.61 ± 0.74^{a} | 5.52 ± 0.74^{a} | <0.0001 |
| Ruminococcus bromii | 1.17 ± 0.31 | 1.14 ± 0.32 | 0.99 ± 0.32 | 0.6421 |
| Eubacterium rectale | 8.78 ± 1.26^{b} | 4.81 ± 1.27^{a} | 4.42 ± 1.27^{a} | <0.0001 |
| Eubacterium hallii | 0.55 ± 0.08^{b} | 0.39 ± 0.08^{a} | 0.30 ± 0.08^{a} | <0.0001 |
| Roseburia spp. | 4.68 ± 0.49^{a} | 4.26 ± 0.50^{a} | 6.45 ± 0.50^{b} | <0.0001 |
| Roseburia intestinalis | 2.49 ± 0.63 | 2.00 ± 0.63 | 2.01 ± 0.63 | 0.4295 |
| Roseburia faecis | 1.27 ± 0.26 | 0.91 ± 0.27 | 0.95 ± 0.27 | 0.0587 |
| Subdoligranulum spp. | 3.28 ± 0.66 | 3.20 ± 0.67 | 0.99 ± 0.32 | 0.6421 |
| Bifidobacterium spp. | 1.90 ± 0.42^{b} | 0.91 ± 0.42^{a} | 1.12 ± 0.42^{a} | 0.0023 |

¹ANOVA with Tukey post hoc test.
²Proportion of bacteria denoted with “spp.” do not include the known members.
^{a,b}Means in the same row not sharing a common superscript letter are different (P < 0.05).

### Example 4

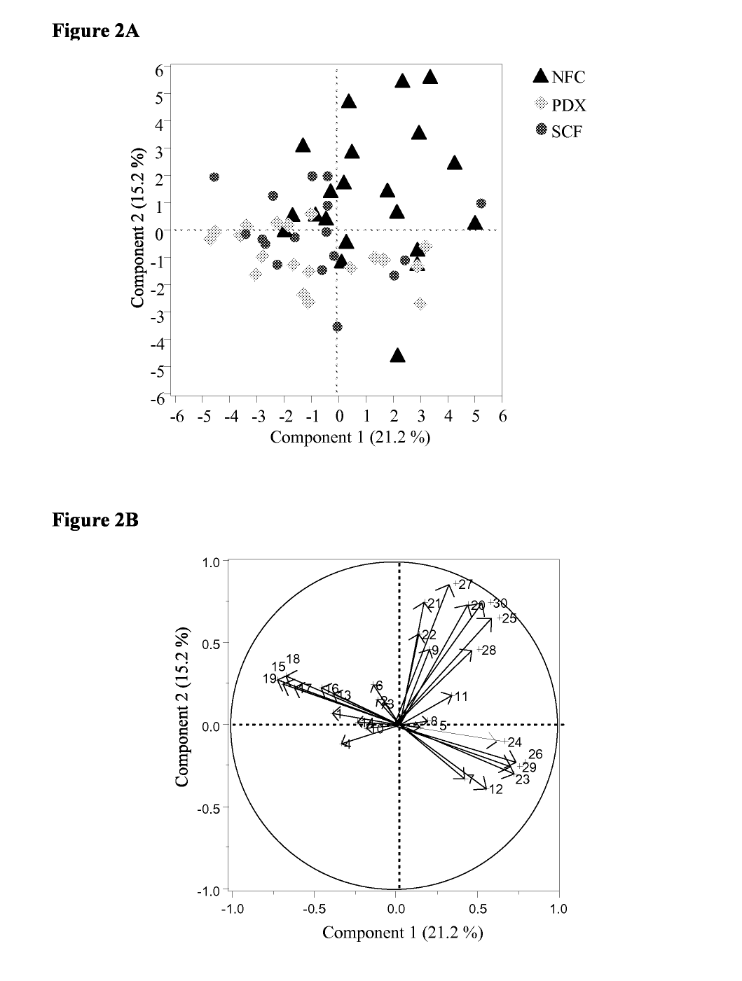

The results of the fecal principal component analysis of Example 1 and the primary bacterial families present in the feces were plotted and are shown as score and loading plots in FIGS. 2A and 2B, respectively. The score plot shown in FIG. 2A shows a distinct separation or clustering of data for the two test groups as compared to the control group. In the loading plot shown in FIG. 2B, the numbered lines represent the following bacterial families: 1, Clostridiaceae; 2, Clostridiales; 3, Bacteroidaceae; 4, Veillonellaceae; 5, Ruminococcaceae; 6, Bifidobacteriaceae; 7, Lachnospiraceae; 8, Eubacteriaceae; 9, Coriobacteriaceae; 10, Alcaligenaceae; 11, Hyphomicrobiaceae; 12, Lactobacillaceae; 13, Fiber; 14, Total intake; 15, Calories; 16, Protein intake; 17, Carbohydrate intake; 18, Total fat intake; 19, Saturated fat intake; 20, Fecal ammonia; 21, Fecal phenol; 22, Fecal indole; 23, Fecal acetate; 24, Fecal propionate; 25; Fecal isobutyrate; 26, Fecal butyrate; 27; Fecal isovalerate; 28; Fecal valerate; 29, Fecal total short-chain fatty acids; 30, Fecal total branched-chain fatty acids.

The data in FIGS. 2A and 2B show the presence of three distinct clusters. The first cluster includes total and individual macronutrient intake (protein, fact, carbohydrates) and fecal Clostridiaceae, Clostridiales, Bacteroidaceae, and Alcanligenaceae and was positively affected by PC2 and negatively affected by PC1. The second cluster includes fecal acetate, proprionate, butyrate, total short chain fatty acid, Lachnospiraceae, Ruminococcaceae, Eubacteriaceae, and Lactobaccilaceae and was positively affected by PC1 and negatively affected by PC2. The third cluster includes fecal branched chain fatty acid, ammonia, phenol, indoles, Hyphomicrobiaceae, and Coriobacteriaceae and was positively affected by both PC1 and PC2.

In further explanation of the results shown in FIG. 2B, various correlations can be seen related to consumption of polydextrose or soluble corn fiber. For example, for the PDX group and the SCF group, an increase in Clostridiaceae, Bacteroidaceae, Veillonellaceae, and/or Alcaligenaceae and/or a decrease in Clostridiales and/or Bifidobacteriaceae was related to and/or caused by an increase in fiber content and/or resistant oligosaccharides. A decrease in Eubacteriaceae, Coriobacteriaceae, and/or Hyphomicrobiaceae as was related to and/or caused by a decrease in ammonia, phenol, indole, isobutyrate, isovalerate, valerate, and/or branched chain fatty acids.

In addition, for the PDX group, a decrease in Ruminococcaceae and/or Lachnospiraceae was related to and/or caused by a decrease in acetate, propionate, butyrate, and total short chain fatty acid content. For the SCF group, a decrease in Ruminococcaceae and/or an increase in Lactobacillaceae was related to and/or caused by a decrease in butyrate and/or an increase in acetate, propionate, and total short chain fatty acid content.

In the foregoing detailed description, the invention has been described with reference to specific embodiments. However, it may be appreciated that various modifications and changes can be made without departing from the scope of the invention. Thus, some of the features of preferred embodiments described herein are not necessarily included in preferred embodiments of the invention which are intended for alternative uses.

1. A method of treating a medical condition comprising ingesting a composition in an amount sufficient to decrease a proportion of Coriobacteriaceae in a gut microbiome of an individual ingesting the composition, wherein the composition comprises polydextrose or soluble corn fiber and wherein the medical condition comprises elevated serum triglycerides, nonalcoholic fatty liver disease, metabolic syndrome, obesity, or type-2 diabetes.

2-3. (canceled)

4. The method of claim 1 wherein the amount of the composition comprises between about 7 grams and 21 grams of soluble corn fiber per day.

5. The method of claim 4 wherein the amount of the composition is consumed daily for between about 1 and about 21 days.

6-8. (canceled)

9. The method of claim 1 wherein the proportion of Coriobacteriaceae is decreased by between about 49 and 87 percent.

10. A method of decreasing serum cholesterol comprising ingesting a composition in an amount sufficient to increase a proportion of Veillonellaceae in a gut microbiome in an individual ingesting the composition, wherein the composition comprises polydextrose or soluble corn fiber.

11. The method of claim 10 wherein the proportion of Veillonellaceae is increased by between about 50 and about 150 percent.

12. (canceled)

13. The method of claim 10 wherein the amount of the composition comprises between about 7 grams and 21 grams of soluble corn fiber per day.

14. The method of claim 13 wherein the amount of the composition is consumed daily for between about 1 and about 21 days.

15-17. (canceled)

18. A method of treating inflammatory bowel disease or Chrohn's disease comprising ingesting a composition in an amount sufficient to increase a proportion of Faecalibacterium, wherein the composition comprises polydextrose or soluble corn fiber.

19. The method of claim 18 wherein the Faecalibacterium comprises F. prausnitzii.

20. (canceled)

21. The method of claim 18 wherein the amount of the composition comprises between about 7 grams and 21 grams of soluble corn fiber per day.

22. The method of claim 21 wherein the amount of the composition is consumed daily for between about 1 and about 21 days.

23. The method of claim 18 wherein the proportion of Faecalibacterium is increased by between about 2 and about 35 percent.

24-26. (canceled)

27. A composition comprising polydextrose or soluble corn fiber in an amount sufficient to decrease a proportion of Coriobacteriaceae or increase a proportion of Veillonellaceae or Faecalibacterium in a gut microbiome of an individual ingesting such composition.

28-29. (canceled)

30. The composition of claim 27 wherein the composition comprises between about 10 percent and about 40 percent polydextrose or soluble corn fiber.

31-33. (canceled)

34. The composition of claim 27 wherein the proportion of Coriobacteriaceae decreases by between about 49 and about 87 percent.

35. The composition of claim 27 wherein the proportion of Veillonellaceae increases by between about 50 and about 150 percent.

36. The composition of claim 27 wherein the proportion of Faecalibacterium increases by between about 50 and about 100 percent.

37. The composition of claim 27 wherein the Faecalibacterium comprises F. prausnitzii.

38. The composition of claim 27 wherein the composition decreases a proportion of Coriobacteriaceae and increases a proportion of Veillonellaceae and Faecalibacterium in the gut microbiome of the individual ingesting such composition.
